(19) United States
(12) Patent Application Publication
(10) Pub. No.: US 2024/0127966 A1
(43) Pub. Date: Apr. 18, 2024

(54) DEVICES, SYSTEMS AND METHODS FOR MONITORING AND PREVENTING THE SPREADING OF CONTAGIOUS DISEASES

(71) Applicant: epyMetrics AG, Zürich (CH)

(72) Inventors: Peter Seitz, Zürich (CH); Yves Delley, Zürich (CH); Edith Schmid, Zürich (CH); Lei Zhang, Zürich (CH)

(21) Appl. No.: 18/546,977

(22) PCT Filed: Feb. 16, 2022
(86) PCT No.: PCT/IB2022/051383

§ 371 Date: Aug. 18, 2023

Related U.S. Application Data
(63) Continuation-in-part of application No. PCT/IB2021/057748, filed on Aug. 24, 2021.
Application No. PCT/IB2021/057748 is a continuation of application No. PCT/IB2021/051413, filed on Feb. 19, 2021.

(30) Foreign Application Priority Data
Feb. 19, 2020 (CH) 00181/20

(51) Int. Cl. G16H 50/80; G16H 50/20

(52) U.S. Cl. CPC G16H 50/80; G16H 50/20

(57) ABSTRACT

Embodiments pertain to an infection detection system comprising a memory; and a processor, wherein the memory and the processor are configured to enable the system to perform the following: receiving physiological data descriptive of physiological parameter values of a subject; receiving non-physiological data relating to the subject; and determining, based on the received physiological data and the non-physiological data, whether at least one infection-detection criterion is met.

### FIELD OF THE INVENTION

The present invention relates in general to devices and systems for monitoring physiological parameters.

### BACKGROUND

Today's long-distance travelling methods by airplane, train, bus and/or ship make a global pandemic due to fast-spreading infections one of the biggest threats to humankind. Since the early detection of infected subjects is difficult, governments react with strict quarantines and severe travel restrictions, causing significant reductions of global travel and trade, which affects the world-wide economy dramatically.

When the human body senses an infection by bacteria or viruses, it reacts with an immuno-response: Special substances are secreted into the blood stream, causing the body to fight the infection. Depending on the specific infection, the immuno-response of the body can elicit particular clinical signs, including, for example, increased temperature (fever), increased sweating, in particular night sweat, coughing, shortness of breath, running nose, sneezing and nasal congestion, sore throat, chills and shivering, increased heart rate, headaches, and/or lowered blood pressure (hypotension).

### BRIEF DESCRIPTION OF THE FIGURES

The figures illustrate generally, by way of example, but not by way of limitation, various embodiments discussed in the present document.

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements. References to previously presented elements are implied without necessarily further citing the drawing or description in which they appear. The figures are listed below.

FIG. 1 is a schematic illustration of an infection detection system, according to some embodiments;

FIG. 2 schematically illustrates an architecture of a wearable device, according to some embodiments.

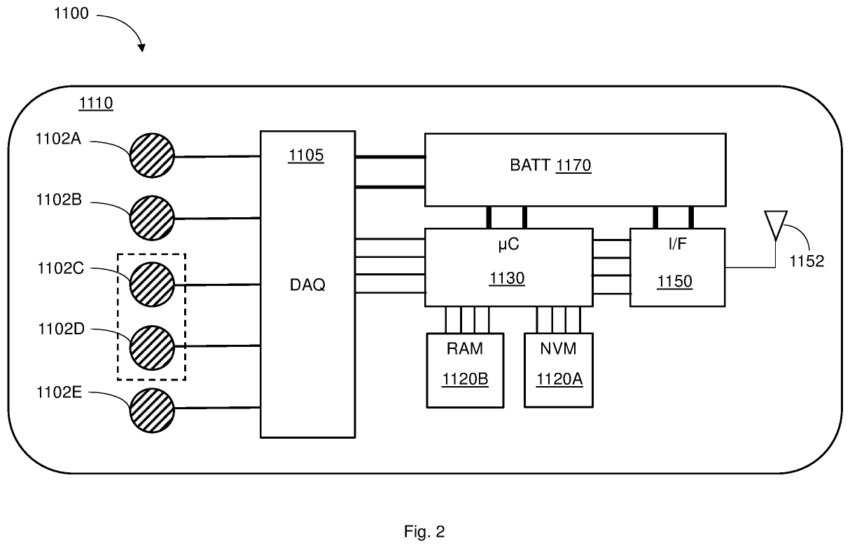

FIG. 3A-B are flowcharts of methods for identifying whether a subject may be affected by an infectious disease.

FIG. 4 is another flowchart of a method for identifying whether a subject is affected by an infectious (e.g., contagious) disease.

### DETAILED DESCRIPTION

Reliable confirmation of an infection requires the detection of antibodies in a patient's blood, that are specific to a certain infection type. This usually necessitates taking blood samples, which can only be carried out by specially trained and certified personnel, requiring also the availability of a correspondingly equipped clinical laboratory.

As a consequence, it is very desirable to have a non-invasive, cost-effective, fast and simple method for the early detection of infections, which can also be administered by untrained personnel. This method is based on recognizing one or several of the clinical signs of an infection listed above.

A wide-spread use for the early detection of infections is the measurement of skin temperature, for example on the forehead or at the wrist of a subject. This method has several disadvantages including, for example:
(1) Only one single measurement is taken, for example at the check-in facility of an airport,
(2) environmental conditions such as, for example air temperature and humidity, at the location where the measurement is made is not taken into consideration,
(3) the individual activity level of a person is not taken into account, for example the elevated skin temperature and increased sweating of a person who had to carry heavy luggage or who had to run to reach the check-in point in time,
(4) the individual differences in the baselines of each vital sign are not taken into account,
(5) the individual circadian (24-hour) temperature rhythms of each person are not taken into account,
(6) the periodic change of temperature at the time of ovulation in females is not taken into account,
(7) no additional clinical signs are taken into account that would allow more accurate interpretation of an unusual temperature measurement.

To overcome the limitations of today's methods for the early detection of infections, aspects of embodiments pertain to an infection detection system that is configured to receive (e.g., through collecting data descriptive of physiological parameter values descriptive of, e.g., vital signs) and, optionally, non-physiological parameter values), with respect to one or more persons or subjects.

Embodiments of the disclosed devices, systems and methods may be configured and employed for monitoring the spreading of contagious diseases.

The system may be configured to analyze the received data to identify data that is descriptive of anomalous physiological parameter values and, optionally, to identify anomalous physiological parameter values that pertain to false positive indications of a clinical signs of a viral or bacterial infection.

In some examples, the system may be configured to distinguish between anomalous physiological parameter values that relate (e.g., correlate) with an event or state that the one or more subjects are affected by an infectious (e.g., contagious) disease, and anomalous physiological parameter values that do not relate (e.g., correlate) with an event or state that the at least one first and/or second subjects are affected by an infectious disease. The distinction may be made through prediction (e.g., classification), for example, by employing a machine learning model including, for example, a neural network, a decision tree, and/or a correlation-based classifier.

Non-limiting examples of clinical signs of a viral or bacterial infection include the following:
(1) increased core-body, rectal and skin temperature, a condition commonly known as fever
(2) increased sweating, in particular night sweat
(3) feeling tired or fatigued
(4) swollen lymph nodes in the neck, armpits, or groin
(5) increased heart rate
(6) headaches
(7) chills and shivering
(8) lowered blood pressure (hypotension)
(9) coughing
(10) shortness of breath
(11) sore throat
(12) running nose, sneezing and nasal congestion
(13) burning or pain with urination.

In some embodiments, when the system senses that physical quantities relating to one or more of the above noted clinical signs are descriptive of anomalous parameter values, then the condition(s) of an infection-detection criterion is or are met. Depending on the clinical sign, a value may be defined or labelled as “anomalous” and output to a user of the system, when the value exceeds a high limit threshold, or when the value drops below a low limit threshold, e.g., for a certain time period. In some examples, a threshold may additionally or alternatively relate to a rate of change of a monitored parameter value.

Thresholds may be static, i.e., predetermined thresholds that remain constant; adaptive thresholds (e.g., adapted depending on environmental factors); or dynamically, i.e., forcefully changed for instance at a certain time of day, and/or a certain day of the week and/or year.

In some embodiments, a threshold relating to a physiological parameter value may be adapted depending, for instance, on environmental and/or healthy physiological baseline values of a subject. For instance, a high limit pulse rate threshold of a trained individual may be set to a higher value than that of an untrained individual.

In some examples, upon the identification of false-positives, the system may determine, e.g., based on a determined probability that an anomalous value is a false positive, whether a respective threshold value shall be altered to reduce probability that the system outputs in the future, under same conditions, false-positives. The system may automatically update the threshold value to an updated threshold value.

In some examples, the system may output (e.g., display) a user an anomalous physiological parameter value along with an indication that that the displayed anomalous value may be a false-positive (e.g., along with a label or with a probability value indicating the probability that the displayed anomalous value is a false positive).

The user may then, via an I/O device of the system, alter the present threshold value related to the parameter value to an updated threshold value to prevent the system from labelling the same value again, under the same conditions, as a false-positive.

In some examples, the system may determine a recommended updated threshold value and provide the user of the system with an output of the recommended updated threshold value. The user may provide the system with an input to indicate acceptance or declining adoption of the recommended updated threshold value.

As outlined herein, the system may be configured to detect or determine a probability that an anomalous parameter value relates to a false positive indication that a subject is affected by an infectious disease. Hence, in some embodiments, an infection detection criterion is not met when, for example, with respect to a certain subject, one or more anomalous parameter values are identified as false positives. In some embodiments, an infection detection criterion is met when none or the minority of the one or more of the anomalous parameter values are identified, e.g., during a certain time period, as false positives. In some examples, a physiological parameter value may be labelled as false positive when the probability that the value is a false positive exceeds a high probability threshold value. In some examples, a physiological parameter value may not be labelled as false positive when the probability that the value is a false positive is below a low probability threshold value.

In some embodiments, under certain conditions, analysis of received data to identify false positives may not be performed by the system. For example, when a subject was diagnosed as being positive for a certain type of infection following a targeted test such as a PCR test, the data characterizing the subject is not analyzed for false positives for a certain time period following the diagnosis (e.g., two weeks). In some examples, the data characterizing the subject and of other subject that were in vicinity of or that came into physical contact with the subject may be analyzed to identify both false-negatives and false-positives.

The most significant clinical signs are (1)-(3), applying to many cases of infection. Additional clinical signs are (4) to (8), which do not appear for any kind of infection. Also, depending on the location of the infection, alternative clinical signs may be observed: An infected wound usually produces clinical sign (4). If the airways and lungs are infected, the clinical signs (9)-(12) may appear. An infection of the urinary tract usually produces clinical sign (13).

Consequently, in some embodiments, generic clinical signs of an infection can include (1)-(3), while the other clinical signs may be used to make a first differentiation of the type of infection, or they may represent the individual reaction of a person to an infection.

Some of the clinical signs of an infection are individual perception, such as (3), (6) or (11), while important clinical signs can be measured accurately with the sensors contained in a wearable device, for example, as described herein may for instance include:
(1) increased temperature can be measured with a skin or core-body thermometer
(2) increased sweating can be measured with a sweat sensor
(5) increased heart rate can be measured with a PPG (photoplethysmography) and/or an ECG (electrocardiography) sensor
(7) chills and shivering can be measured with an accelerometer
(8) lowered blood pressure (hypotension) can be measured with a PPG, whose signals are exploited for the determination of systolic and diastolic blood pressure
(9) coughing can be measured acoustically, with a microphone or an accelerometer
(12) sneezing can be measured acoustically, with a microphone or an accelerometer.

Additional information about the possible source of an infection can be gained by analyzing a patient's blood for the following analytes:
(A) C-reactive protein (CRP)
(B) Procalcitonin (PCT)
(C) Lactate
(D) Bilirubin, creatinine, platelets (SOFA score)
(E) White blood cell count (WBC)
(F) Absolute neutrophil count (ANC)
(G) Erythrocyte sedimentation rate (ESR).

Reliable confirmation of a source of an infection requires the detection of antibodies in a patient's blood that are specific to a certain infection type.

Healthy base-line values of the vital signs that can be measured with the seven sensor types mentioned below depend on the individual physiology of a patient, the degree to which he/she is a trained sportsperson, and the individual acclimatization of a person to its environment.

As a consequence, it may be highly desirable that a person is using his/her wearable from time to time, in order to acquire the subject's rest values of the various vital signs. Also, the wearable device should be attached as soon as a first indication is obtained that there might be an infection, because the temporal evolution of the vital signs provides a key insight into the development of an infection or in determining that the occurrence of an infection can be eliminated.

In some examples, rest values or healthy baseline values of the various vital signs can be defined as follows:
(1) Core-body temperature of between 37.5° C. and 38.0° C. Acclimatization to changed working or climatic environments may lower core-body temperature by more than 0.5° C.
(2) Rest values for sweat rate depend on the measurement location (locations may differ by a factor of up to 20), on the gender (females are producing less sweat than a male), on the training condition (trained sportsmen are sweating more than untrained persons), on the body surface area, and on the climatic conditions: Water loss from cutaneous evaporation could range from 500 ml per day at rest in a cool environment to 10 liters per day during exercise in the heat.
(5) Rest heart rate (RHR) of a not-well trained adult is between 60-100 beats per minute (bpm) of an adult. Well-trained sportsmen have a lower HRH of typically 40-60 bpm.
(7) Shivering can be recorded as involuntary, periodic muscle contractions with a frequency above 1 Hz. Under healthy, normal conditions (the person is feeling warm), no shivering is present.
(8) Diastolic blood pressure in healthy individuals is below 80 mm Hg, and systolic blood pressure is below 120 mmHg. Significant daily variations can be observed in rest blood pressure, and it is therefore important to provide long-term observations of the blood pressure trends.
(9) Coughing can be detected according to the acoustic signature of body noise, containing acoustic frequencies above 10 Hz. Healthy individuals are typically not coughing; they may only clear their throats from time to time, with a frequency not exceeding about 10 times per hour.
(12) Sneezing can be detected according to the acoustic signature of body noise, containing acoustic frequencies usually higher than that of coughing.

With regards to sweat, proto sweat is interstitial fluid, i.e., the liquid surrounding the cells in organs and tissue. For this reason, analytes found in blood are also found in interstitial fluid, and therefore probably also in sweat. For larger analytes it is often more difficult to pass the blood-tissue barrier, and they may therefore by found in lower concentrations in sweat compared to blood. Also, larger cells such as erythrocytes are not found in sweat. Thus, the following infection biomarkers may be found in sweat:
(A) C-reactive protein (CRP)
(B) Procalcitonin (PCT)
(C) Lactate
(D) Bilirubin and creatinine (incomplete SOFA score)

In some embodiments, as mentioned herein, the infection detection system includes one or more wearable devices with which relevant vital signs of a user can be determined continuously and non-invasively, thus following a subject's health state during, for example, several hours or days. In this way, signs of an incipient infection can be identified, for example, during the time of travel, for example by airplane, by train, by ship and/or by bus, and/or during quarantine for subjects arriving from an infected region, independently of the differences in the individual physiology of each user.

Furthermore, the embodiments relate to a method of (e.g., automatically) associating (e.g., correlating) external, environmental conditions with the physiological response of a person, thus enabling differentiating between disease-associated physiological signs of an infection and normal physiological signs occurring due to natural physiological responses of a subject to varying environmental conditions. In some examples, tracking of physiological and/or non-physiological parameter values (e.g., location-based tracking) of a subject of may allow differentiating between occurrence of sweating due to psychological stress (e.g., while sitting an exam) from sweating due to overheating.

For example, in case elevated body temperature of a subject is found to correlate with extensive physical activity under environmental harsh conditions, then the physiological sign of elevated body temperature may not necessarily provide an indication that the person is affected by an infectious disease. Accordingly, the confidence level that the physiological sign of elevated level temperature is indicative that the subject is affected by an infectious disease may be lowered. On the other hand, in case a subject has elevated body temperature, although the environmental conditions and/or the subject's activity are not associated with elevated temperature, then the subject's elevated body temperature may be found to be more likely associated with clinical signs that the subject is affected by an infectious disease. For example, if a subject is located over a prolonged period of time (e.g., several hours) in a room at 20-22° C. and the subject has elevated body temperature, although the subject is not pursuing any strenuous physical activity, then the clinical sign of elevated body temperature is more likely to be indicative that the subject is affected by an infectious disease, at comparatively increased confidence interval.

Embodiments may also pertain to long-term monitoring of patients who have been treated for a medical infection, and whose reactions to the medical treatment can be followed continuously after discharge from a hospital or from a doctor's practice. For example, embodiments may pertain to continuously monitoring a patient's vital signs related to infections after the person has been treated at a hospital, at a doctor's practice or at another point of medical care. This out-patient monitoring can occur during quarantine, at work, at home and/or in transit.

Embodiments may further pertain to provide a device, system, and a method for the reliable, cost-effective, fast, simple and early detection of infections, in particular of (comparatively fast spreading) infections that could develop into an endemic or pandemic outbreak, for example, by continuously monitoring a person's vital signs related to infections during an extended time, in particular during the period a person is travelling. For example, the device, system and method may allow the early detection of infections and are compatible with today's easily accessible means of long-distance travelling, for example, by airplane, by train, by ship or by bus.

Aspects of embodiments pertain to an infection detection system comprising a wearable device. The wearable device may be operably engaged at one or more locations of the person's body surface including, for example, the forehead, torso and/or forearm.

The wearable device may include at least one sensor (e.g., employed by a wearable device) sensors for the measurement of the activity level of a person, such that this vital sign can be stored for further processing at each point in time.

The infection detection system may receive and process data relating to and/or descriptive of a person and/or its activities, all of which may herein also be collectively referred to as “a state of the subject”. The data received and processed by the system may for example pertain to physiological characteristics (e.g., vital signs) of the person, menstrual cycle which may influence a female's body temperature, circadian rhythms of the subject (e.g., circadian temperature and/or pulse rhythm), and/or non-physiological characteristics including, for example, environmental characteristics in which the person is located, descriptive of a route traversed by the person and/or a type of body motion and/or posture, jet lag, and/or the like. Jet lag alters the circadian rhythms of a subject which, in turn, may influence various physiological parameters.

In some embodiments, data about a person being monitored may be received from sensors worn by the person (“body-worn sensors”) and/or from external databases such as healthcare management databases, weather information databases, social media, (e.g., environmental) sensors on board of the transportation means, and/or the like.

In some embodiments, the infection detection system may comprise at least one physiological sensor for sensing or measuring one or more physiological characteristics of the person. In some embodiments, the infection detection system may comprise a wearable device comprising the at least one physiological sensor. The infection detection system and/or the wearable device may be configured to measure or sense physiological parameter values with the at least one sensor over an extended period of time (tens of minutes, several hours or during several days).

Physiological characteristics measurable by the at least one physiological sensor may include, for example, systolic blood pressure, diastolic blood pressure, mean arterial pressure, pulse rate, breathing rate, breathing pattern, coughing patterns, coughing types (e.g., dry or wet coughing), oxygen saturation level, glucose level, electrical property of the patient's skin (e.g., conductivity, resistance), weight, body-mass index (BMI), pH level, concentration of one or more selected or predetermined analytes in bodily fluid (e.g., magnesium, calcium, natrium, salts, glucose, and/or hormones), motor function, body temperature, sweat rate, electrocardiogram, myocardiogram, electroencephalography (EEG), capnography values, gastro-intestinal (GI) tract activity sensor (e.g., based on sound), body odor, and/or cognitive ability of the patient. Bodily fluids may include, for example, blood, sweat, tears, urine, and/or saliva. In some embodiments, sensors may also be employed to analyze feces. Further physiological parameter values such as blinking of eyes, etc., may provide an indication on how fatigued the subject is. In some instances, physiological signs of infection that are difficult to measure with a wearable can be requested from the traveler, for example whether he or she is suffering from increased temperature (fever), increased sweating, in particular night sweat, coughing, shortness of breath, running nose, sneezing and nasal congestion, sore throat, chills and shivering, increased heart rate, headaches, and/or lowered blood pressure (hypotension).

In some example, sore throat, nasal congestions and/or the like, may be detected through the employment of auditory sensors (microphones) for recording voice outputs, coughing activities and/or sneezing, voluntarily or involuntarily provided by the subject being monitored.

The infection detection system is further configured to receive data descriptive of non-physiological information such as environmental characteristics in which the person is located. For example, the infection detection system may comprise at least one sensor for sensing and/or measuring, with at least one environmental sensor, values related to environmental characteristics such as ambient temperature, humidity, pressure and/or lighting conditions of the environment in which the person is located.

The infection detection system may further be configured to determine whether the person is located outdoors or indoors. In case it is determined that the person is located outdoors, additional environmental characteristics such as precipitation and/or cloudiness may be sensed. In some embodiments, the wearable device may comprise the at least one environmental sensor.

In some embodiments, the infection detection system may be configured to determine (including, e.g., estimate) the person's position within a reference coordinate system (e.g., the world coordinate system), for example, based on a space-based global navigation satellite system (e.g., the US-based Global Positioning System). For example, the wearable device, which may be worn by the person, may comprise a receiver device configured to receive satellite navigation signals based on which a position of the receiver device may be determined, along with a corresponding time stamp. In some embodiments, a person's position may be estimated independent of a space-based global navigation satellite system. For instance, a person's position may for example be determined based on Visual Simultaneous Localization and Mapping (SLAM) techniques.

In some embodiments, the infection detection system may comprise at least one body motion sensor for determining a type of motor activity undertaken by the subject. Types of motor activity can include, for example, running, walking, ascending stairs, descending stairs, jumping, sleeping, eating, standing, seating, shivering, swimming, (e.g., scuba) diving, skiing, etc. Additional physical types of motor activity may relate to performing physical labor (e.g., operating a jack hammer, mining-related activities, etc.). The at least one sensor may further be employed to determine a breathing rate, a breathing pattern, and/or the like. For example, one or more inertial sensors and/or magnetometers may be employed for determining a person's type of motor activity he/she is engaging with, a breathing rate, a breathing pattern, etc., e.g., through the processing of signals received from the sensors employed by the infection detection system. In some embodiments, infection detection system may be configured to perform balance evaluation of the person.

In some embodiments, the infection detection system may be configured to assess or evaluate a subject's neurological activity for detecting, for example, a neurological deficit. Neurological activity may for example be determined through EEG measurement, measurement of motor and/or somatosensory parameter values, and/or the like.

Data about the person may be brought in association with each other (e.g., through correlation) for determining the subject's physiological characteristics, environmental conditions to which the subject has been subjected, the person's expected travel itinerary, the person's medical background, and/or the like, over a period of time, to derive a conclusion regarding the person's health status during this period of time.

For example, physiological data descriptive of an infectious disease (clinical-indication data) may be complemented (e.g., through correlation) with data (also: complementary or non-clinical data) descriptive of the person and the person's activity. Non-clinical data may be descriptive of information that is not, per se, considered to provide an indication concerning a person's immune response to an infectious disease.

The system may process the infection data along with the complementary data to reduce the probability or rate of false-positive diagnostics of an infectious disease and/or to reduce the probability of false-negative diagnostics. For example, elevated air temperature, humidity, physical activity, times when a meal is served may be taken into consideration to identify false positives. Periods during which a person is asleep and/or the light is dimmed (e.g., dimming of light in airplanes, cinemas, theaters), may be taken into consideration (e.g., processed by the system) for determining a likelihood that a person having nevertheless elevated (e.g., abnormal) body temperature, is indeed affected by or is suffering from (e.g., has contracted) a viral and/or bacterial infection.

Considering the vast number of activities different persons may be involved in at any given point in time and further considering the subjects' different medical and physiological backgrounds, it may be difficult to conclusively establish that one person having, for example, elevated body temperature is indeed suffering from or affected by an infectious disease. On the other hand, the fact that one person does not have elevated body temperature does not necessarily suffice to conclusively establish that the same person is not affected by or suffering from an infectious disease.

To increase the level of confidence of an analysis output indicating whether a person is affected or not affected by an infectious disease, the infections detection system may be configured to associate subjects with each other to establish a cohort (also: group) under investigation and monitor at least two of a plurality of subject members of the cohort.

In some embodiments, the infection detection system may be configured to automatically identify subjects suitable for inclusion in a cohort, for example, based on the subjects' activity. In some embodiments, the infection detection system may be configured to automatically identify subjects to be excluded from a cohort.

The inclusion in and exclusion of subjects from a cohort may for example be location-based and/or activity based.

Associating a plurality of persons to define a cohort may be accomplished in several ways. For example, the movements of persons may be tracked (via, the receiver of their wearable devices) to identify clusters or similar patterns in movement. For example, the infection detection system may be configured to perform cluster analysis on data which is descriptive of estimated location points received from receiver devices worn by a plurality of subject. The estimated location points may be associated with time stamps of recordation of the estimated location points. The plurality of estimated location points may be grouped by clustering to obtain data clusters, according to the geographic distribution of the location points as a function of time. For example, for a given time stamp, a group of location points may be associated with a cluster if they are distributed in a certain geographic area within a relatively higher density than other location points. Cluster analysis techniques that may be employed may include, for example, Density-based Spatial Clustering of Applications with Noise (DBSCAN), K-Means, or the like.

This way, subjects which are pursuing, or which are expected to pursue similar activities within a geographic region or area may be associated with each other in a cohort.

For example, persons who are identified through clustering as using a same commuting route within a certain time of day may be associated with a same cohort. In another example, passengers and crew boarding a particular flight may be associated in a cohort.

The evolution of clinical signs recorded for a plurality of persons in the same cohort can be followed as a function of time, and correlated with non-infection data such as, for example, external measurement conditions and the person's individual activity level.

Infection data as well as complementary data of a plurality of subject members of the same cohort may be received and processed by the infection detection system to determine, based on at least one infection-detection criterion (e.g., by determining a level of correspondence and/or by correlating between clinical signs recorded for a plurality of persons in the same cohort with non-infection data), which member of the cohort is affected by or suffering from an infectious disease and/or which person is not affected by or suffering from an infectious disease. In some embodiments, the infection detection system may output values indicating (e.g., a probability) that a person is or is not affected by an infectious disease. The outputs provided by the system may be of numerical, ordinal and/or categorical type. The expression infection-detection criterion may be defined, for example, by one or more thresholds relating to measured values of physiological and non-physiological characteristics.

In some embodiments, the movement of at least one subject may be tracked for the purpose of investigating human behavior to derive epidemiological conclusions and/or to provide behavioral recommendations and/or to disrupt or interrupt a chain of infections. For example, the detection infection system may provide travel recommendations to mitigate, reduce the risk or prevent the spread of infections. For instance, different cohorts and/or different subjects may be provided with different travel journey recommendations such that for example a first cohort having a member identified as being infected does not come in contact with another cohort that does not have an infected member.

For example, the infection detection system may monitor at least two subjects of at least one first cohort associated with or expected to travel along a first initial route from a first origin to a first destination, and at least two subjects of at least one second cohort associated with or expected to travel along a second initial route from a second origin to a second destination. The first and the second routes may at least partially overlap in time and location (e.g., share a same stopover location and/or common means of transportation for at least a part of the route). Based on the data gathered and processed with respect to the at least two subjects of each cohort, the system may identify a first subject member of the at least one first cohort as being affected by (e.g., having contracted) an infectious disease. The at least one first cohort may thus become “flagged”, and the corresponding routes may be automatically altered by the infection detection system to redirect the at least one first and/or the at least one second cohort along a first and/or second new route such as to isolate the at least one first cohort from the at least one second cohort during their journey. In some examples, the first and/or the second origin may be identical or different from each other. In some examples, the first and/or the second destination may be identical or different from each other. It is noted that the above example of redirecting a cohort from an original route may also be applicable on an individual level and applied, for example, to subjects traveling with their cars, taxis and/or ride-sharing services. For example, cab drivers, drivers of cars or other vehicles utilizing ride-sharing services, and/or drivers who provide chauffeur services, may be alerted about customers that are identified as (likely) having contracted an infectious disease, and may be diverted to other “non-infected” customers.

In some embodiments, infection detection system is configured to acquire or receive, during an extended time period, data pertaining to physiological and/or non-physiological parameter values of one or more subjects, for example, for determining a state of the one or more subjects. Based on the data, the infection detection system may identify an infection, predict an onset of an infection and/or identify an incipient infection. Based on prolonged monitoring of one or more subjects, a potential infection can be identified with higher certainty at the end of each person's journey, overcoming the above-mentioned shortcomings of today's methods.

With the foregoing objects in view, embodiments pertain to a wearable device comprising one or more sensors configured to produce, over a period of time, responsive to sensed physical stimuli pertaining to a physiological parameter value of the subject, processable electronic signals. The electronic signals are processed for the purpose of detecting clinical signs of an infection by which the subject may be affected or may become affected.

This wearable device is configured to be worn at a location on the body where the clinical signs such as core body temperature, acoustic signals, accelerations and/or sweat-related characteristics (e.g., sweat rate, concentration of analytes in the sweat) can be reliably measured. During an extended time—from tens of minutes to a few days—these clinical signs are acquired such that these measurements can be correlated with external conditions, for example air temperature and humidity. In addition, the wearable device contains sensors for the measurement of the activity level of the user. The device is typically worn during the time of travel, such that the temporal evolution of the clinical signs can be tracked during a journey.

In this way, individual physiological baselines, baseline shifts, circadian rhythms, environmentally induced and/or activity-caused variations of the clinical signs of an infection can be taken into account, and a more reliable identification and possibly prediction of an onset of an infection can be given. Predicting the probability of becoming subjected to or suffering from an infectious disease may be helpful to reduce the spread of an epidemic or pandemic disease, to reduce the time a person has to spend in quarantine, to follow up on a medical treatment, to recall an endangered patient to the treating point-of-care (e.g., hospital, a medical practice), and/or as monitoring of the recovery process of a hospital out-patient or ambulatory patients.

As mentioned herein, baseline values of the vital signs of healthy persons can show large inter-individual differences, and the baseline values of the same person can vary from day to day. It is therefore important to know the mid-term evolution of each user's vital signs, to be able to deduce what an individual “healthy range” is.

One of the most reliable generic indication of a potential infection is the core-body temperature (CBT). If a healthy, not well-trained individual at rest in a cool environment (18-22° C.) exhibits a CBT of more than 38° C., he/she may be running a fever because of an impending infection. In that case, an infection detection criterion may be met.

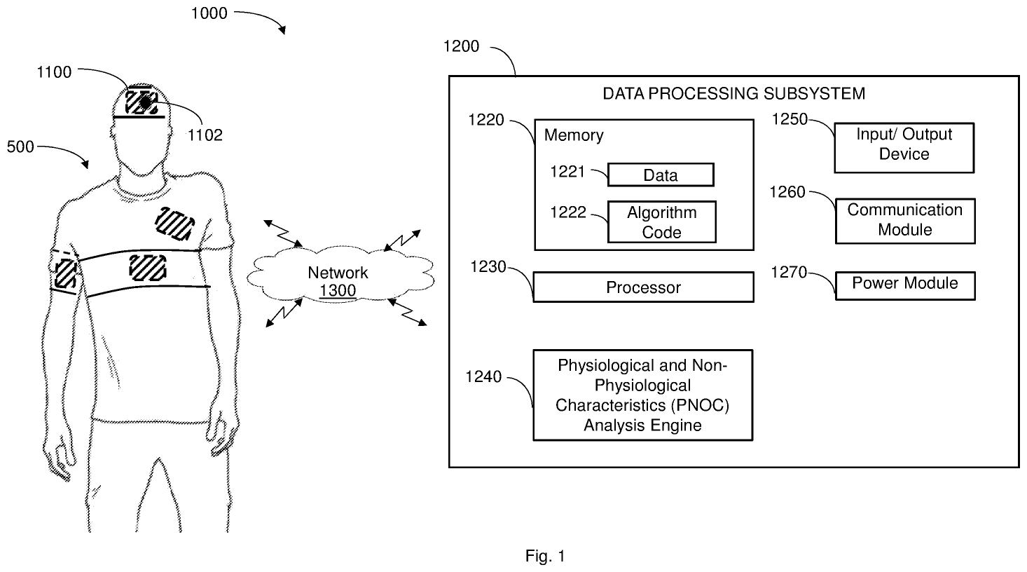

Reference is made to FIG. 1. According to some embodiments, an infection detection system 1000 comprises one or more wearable devices 1100 that can be worn by a subject 500, and a data processing subsystem 1200. In some embodiments, one or more components and/or modules of data processing subsystem 1200 may be part of wearable device 1100. In some other embodiments, one or more components and/or modules of data processing subsystem 1200 may be external to wearable device 1100.

Wearable device 1100 may comprise at least one or a plurality of sensors 1102 which are configured to generate processable signals, responsive to being subjected to physiological and/or non-physiological physical phenomenon and/or stimuli. Hence, the signals generated by the at least one sensor 1102 may relate to or descriptive of sensed physical phenomenon.

Data relating to or descriptive of physiological and/or non-physiological signals generated and/or received at data infection system 1000 may be stored in a memory 1220 for processing by a processor 1230. Memory 1220 may be configured to store software such as data 1221 and/or algorithm code 1222 (e.g., software of rule-based algorithm codes and/or machine learning models) for the processing of physiological and/or non-physiological data of one or more subjects resulting in the implementation of a Physiological and Non-Physiological Characteristics (PNOC) Analysis Engine 1240.

It is noted that although certain functionalities of infection detection system 1000 are described herein with respect to wearable device 1100, this should by no means be construed as limiting. Accordingly, in some embodiments, functionalities of infection detection system 1000 may be implemented fully or partially by a multifunction mobile communication device also known as “smartphone”, a mobile or portable device, a non-mobile or non-portable device, a digital video camera, a personal computer, a laptop computer, a tablet computer, a server (which may relate to one or more servers or storage systems and/or services associated with a business or corporate entity, including for example, a file hosting service, cloud storage service, online file storage provider, peer-to-peer file storage or hosting service and/or a cyberlocker), personal digital assistant, a workstation, a wearable device, a handheld computer, a notebook computer, a vehicular device, a non-vehicular device, a stationary device and/or a home appliances control system. For example, some functionalities PNOC analysis engine 1240 functionalities may be implemented by wearable device 1100, some by devices and/or systems external to the wearable device. Alternative configurations may also be conceived.

Infection detection system 1000 may further include an input/output device 1250. Input/output device 1250 which may be configured to provide and/or receive any type of data or information, for example, from an operator of the system, subject 500 being monitored, and/or other authorized personnel. Input/output device 1250 may include, for example, visual presentation devices or systems such as, for example, computer screen(s), head mounted display (HMD) device(s), first person view (FPV) display device(s), device interfaces (e.g., a Universal Serial Bus interface), and/or audio output device(s) such as, for example, vibrator(s), speaker(s) and/or earphones. Input/output device 1250 may be employed to access information generated by the system and/or to provide inputs including, for instance, control commands, operating parameters, queries and/or the like. For example, input/output device 1250 may allow a user of infection detection system 1000 to perform one or more of the following: approval to start tracking movements of a subject; viewing outputs provided by the system related to physiological and/or non-physiological data; performing queries; providing data input; approval or disproval of a subject inclusion in or exclusion from a cohort; defining cohort inclusion and/or exclusion conditions; a system operating mode (e.g., automated mode, semi-automated mode for determining cohort parameters values); defining a subject anonymization level (full-anonymization level; partial anonymization level; non-anonymous level).

Anonymization could relate to the person himself/herself, to his/her gender, to his/her activities, to his/her localizations and/or to his/her medical conditions.

In a non-anonymization level, personal data of the subject may be made available for inspection by third parties. In a partial anonymization level, information about subjects may be anonymized at the cohort level. For example, only cohort related information about subjects may be made available to third parties. In a full-anonymization level, cohort information may also be anonymized.

Infection detection system 1000 may further include at least one communication module 1260 configured to enable wired and/or wireless communication between the various components and/or modules of the system and which may communicate with each other over one or more communication buses (not shown), signal lines (not shown) and/or a network infrastructure. RF-based wireless communication; optical-based wireless communication such as infrared (IR) based signaling, and/or wired communication.

Network 1300 may be configured for using one or more communication formats, protocols and/or technologies such as, for example, to internet communication, optical or RF communication, telephony-based communication technologies and/or the like. In some examples, communication module 1260 may include I/O device drivers (not shown) and network interface drivers (not shown) for enabling the transmission and/or reception of data over network 1300. A device driver may for example, interface with a keypad or to a USB port. A network interface driver may for example execute protocols for the Internet, or an Intranet, Wide Area Network (WAN), Local Area Network (LAN) employing, e.g., Wireless Local Area Network (WLAN)), Metropolitan Area Network (MAN), Personal Area Network (PAN), extranet, 2G, 3G, 3.5G, 4G, 5G, 6G mobile networks, 3GPP, LTE, LTE advanced, Bluetooth® (e.g., Bluetooth smart), ZigBee™, near-field communication (NFC) and/or any other current or future communication network, standard, and/or system.

Infection detection system 1000 may further include a power module 1270 for powering the various components and/or modules and/or subsystems of the system. Power module 1270 may comprise an internal power supply (e.g., a rechargeable battery) and/or an interface for allowing connection to an external power supply.

It will be appreciated that separate hardware components such as processors and/or memories may be allocated to each component and/or module of infection detection system 1000. However, for simplicity and without be construed in a limiting manner, the description and claims may refer to a single module and/or component. For example, although processor 1230 may be implemented by several individual processor cores or chips distributed at various locations, the following description will refer to processor 1230 as the component that conducts all the necessary processing functions of infection detection system 1000.

A wearable device 1100 comprises flexible or rigid substrate 1110, on which various sensors 1102 are placed. Substrate 1110 may be encased in a housing configured to allow operably engaging wearable device 1100 with a subject for a comparatively prolonged period of time (e.g., hours, days, weeks or even months).

The most important vital sign indicative of infection is the temperature (skin temperature and core body temperature), and therefore special care must be taken that temperature measurement is comparatively accurate. It is known that temperature readings on the human body are dependent on outside conditions and the exact location of the temperature measurement on the body: If it is cold outside, temperature readings at the periphery (hands, wrists, arms, feet, legs) can be significantly lower than the core body temperature measured at the torso or head. Temperature readings on the torso and on the forehead remain much less dependent on ambient conditions. Example measurement sites are schematically shown in FIG. 1 and can include the subject's torso, forehead, and upper arm. Further example measurement sites, although possibly providing less reliable readings, can include the subject's wrist, ankle, etc.

Wearable device 1100 may be operably engaged with subject 500 by employing one or more fasteners such as, for example, straps, belts, cuffs, (e.g., hypoallergenic) adhesives, and/or the like.

Sensors 1102 may include non-inertial sensors and inertial sensors employed for sensing and recording of physiological and/or non-physiological parameter values. Inertial sensors may include, for example, one or more accelerometers (angular and/or linear) and/or gyroscopes.

Sensors 1102 may include and/or be employed for the implementation of, for example, one or more barometers, cameras and/or magnetometers (e.g., for indoor and/or outdoor tracking of movement), proximity sensors, altimeters, light sensors, body temperature measurement devices, oximeters, glucose meters, pulse rate measurement, blood pressure measurement, glucose level in blood, skin conductivity, sweat rate, secreted bodily fluid analysis subsystems, a type of cough (e.g., dry or wet cough), EEG activity measurement, GI activity measurement, receivers of a Satellite-based Positioning System, a type of physical activity, levels of activity of the subject, sneezing, shivering and/or, chills (e.g., through the analysis of on-body accelerometers and/or microphones acting as stethoscopes), the sensing of ventilation-related parameters (e.g., a breathing rate, breathing volume) for the detection of abnormal ventilation or breathing such as Hyperventilation, Dyspnea, Bradypnea, Tachypnea, (sleep) Apnea, and/or the like; for determining a type of cough (e.g., dry or wet), and/or a type of sneezing (e.g., due to a cold or due to an allergic reaction) and/or the like. Sensors 1102 that may be employed for the sensing of ventilation-related parameters values may include, for example, acoustic sensors, inertial sensors, strain sensors. Strain sensors may for example be incorporated in flexible belts straps and/or elongated patches for measuring breathing-related parameter values, for example, through substrate elongation. The flexible belts and/or straps may be employed as fasteners to fasten wearable device 1100 on or otherwise operably engage the device with the body of subject 500.

Sensors 1102 such as inertial sensors and/or cameras (e.g., street cameras) may be employed to monitor a subject to determine, based on sensor signals provided by sensors 1102, a state (resting state or non-resting state) of the monitored subject to determine whether the subject is in a resting state (e.g., sitting in a train) or in a non-resting state (e.g., exercising, running to catch a bus or train, etc.) and/or determine whether the subject is engaging in a strenuous activity or not. Temporal evolution of the monitored vital sign signal(s) may be analyzed to determine whether the subject's state is transitioning from a resting state to a non-resting state, or vice versa.

For example, in case a monitored movement or motion of the subject indicates that the subject is transitioning from a non-resting state to a resting state (e.g., cameras and/or accelerometers indicate that the subject has run to the train and is now sitting in the train), and vital sign signals such as pulse and body temperature begin to correlate with the monitored transition over time, then measured elevated (e.g., anomalous) body temperature, elevated (e.g., anomalous) sweat rate, and/or elevated (e.g., anomalous) pulse can be disregarded as an indication that the subject is affected by an infectious disease.

In some examples, a transitional time period, measured from a time point where the resting-state starts, may be taken into consideration to determine whether anomalous vital sign parameter values return to a normal range, and/or the rate at which anomalous vital sign parameter values return back to fall within the normal range of the monitored subject. Provided that the subject remains in the resting state for some time exceeding the transitional time period, and if during or shortly after the transitional time period (e.g., up to 5 minutes) thereafter, the vital signs are still measured within anomalous ranges, it may be derived that the subject is affected by an infectious disease. That is, because even though the transitional time period has passed, the vital signs values still have not returned to be within their normal range. In such case, the subject's activity may be determined as not correlating well (e.g., is below a correlation coefficient threshold) with the monitored vital signs and, e.g., the probability of a false-positive diagnostics of an infectious disease is comparatively low. In that case, the infection detection criterion can be considered to be met.

On the other hand, when a monitored transition from one state (e.g., non-resting state) to another state (e.g., resting state) does correlate well (is above a threshold correlation) with monitored vital signs, for example, when the vital signs value are anomalous during the non-resting state yet slowly return during the transitional time period to their normal range, then it may be determined that measurements taken in the non-resting state do not provide an indication that the subject is affected by an infectious disease, e.g., the probability of a false-positive diagnostics is comparatively high. In this case, an infection detection criterion is not met.

Additional or alternative parameter values that may be monitored for determining a probability of a false-positive determination of an infectious disease can include, for example, unusual environmental conditions in the resting place (too cold, i.e., temperature is below a low limit threshold, or too hot, i.e., environmental temperature is above a high limit threshold, or too humid, e.g., a relative humidity is above a high limit threshold), which may lead to excessive sweating or possibly increase of CBT; and/or psychological stress, which is known to increase sweat rate, heart rate, skin temperature and possibly core-body temperature.

Psychological stress maybe quantified, for example, by determining the concentration of the stress hormone Cortisol in the sweat. Increase of the metabolic activity of a person, can influence sweat rate, heart rate, skin temperature and possibly also core-body temperature. This may be caused by physical activity (detected through accelerometer monitoring) but it may also be due to digestion of a meal. This could be quantified, for example, by monitoring the glucose concentration in sweat (which is known to track the glucose concentration is blood accurately), by monitoring a gastrointestinal activity (bowl sound, electrophysiological signals characterizing GI activity, etc.)

In some embodiments, sensors 1102 may be configured to determine body surface temperature, thermal flux and/or core body temperature, e.g., by making direct or indirect mechanical and/or thermal contact with the skin.

In some embodiments, sensors 1102 may include and/or be employed by photo-plethysmography (PPG) systems for measuring, for example, heart rate, heart rate variability, cuff-less measurement of blood pressure and/or the detection of hypotension.

In some examples, sensors 1102 may employ optical measurements techniques, requiring a free line-of-sight (LOS) to the skin for shining light into the human body and for collecting the back-scattered light for analysis, and/or other measurement techniques that may be based on electromagnetic (EM) radiation and which may not necessarily require a free LOS between an EM radiation emitter and EM radiation sensor. In some examples, sensors 1102 employing optical techniques may be employed for the measurement of the oxygen saturation of the blood (e.g., SpO2 sensors). In some embodiments, sensors 1102 may include multi-wavelength reflectometers based on which, for example, the hydration, fat content, and/or vascularization of a tissue region can be estimated.

Further types of sensors 1102 may be operable to output bio-impedance signals for determining the electrical conductance of the tissue at various frequencies, providing for example information about the hydration, fat-content and/or vascularization state of the tissue.

Further types of sensors 1102 require free access to the fluid or gaseous environment over the skin for the measurement of the sweat rate. For example, sensor 1102D may collect sweat produced by the sweat glands on the skin surface, for analysis in sweat-analysis microsystems. Depending on the sensitivity and the selectivity of the employed sensors, various ions and molecules related to infections can be detected in the sweat, and/or in other bodily fluids.

Sensors 1102 may produce analog or digital signals. In either case, the signals are read out and converted into digital data samples with electronic data acquisition (DAQ) subsystem 1105 of wearable device 1100. Physiological data descriptive of physiological parameter values are stored along with a time stamp, so that each data point can later be associated with non-physiological parameter values relating to, for example, external conditions and/or events.

In some examples, DAQ subsystem 1105 is operably coupled with a microcontroller or microprocessor 1130 that is powered by a battery 1170.

Microprocessor 1130 is connected to non-volatile memory (NVM) 1120A and random-access memory (RAM) 1120B. Non-volatile memory 1120A may contain the program for processor 1130, serial numbers, calibration data, etc. In some examples, a memory of system 1000 such as random-access memory 1120B may contain intermediate values employed for the calculation of calibrated data, it may contain the parameters of a physiological model of the wearer (a so-called digital twin) to identify deviations from what may be considered “normal behavior”. The memory further stores all the acquired data obtained, for example, from sensors 1102A-1102E, until this data is read out.

Readout of data occurs through interface (IF) 1150, which is either employing a wire-based communication technology such as the Universal Serial Bus (USB), or a wireless communication technology such as, for example, Bluetooth or LTE Cat-M1. In the case of a wireless communication technology, at least one antenna 1152 may be employed for exchanging data bidirectionally systems external to wearable device 1100.

It is noted that the architecture of wearable device 1100 described herein should by no means be construed in a limiting manner. Accordingly, additional, or alternative configuration may also fall within the scope of the present invention.

According to some embodiments, wearable device 1100 is configured such that it can be operably engaged with a subject for a comparatively extended period of time (e.g., hours or even days) at locations which are be considered to provide comparatively more reliable readings of reference and/or base values.

In some embodiments, infection detection system 1000 may employ a plurality of wearable devices 1100. In some embodiments, one wearable device may be used as reference for another wearable device, for example, for calibration purposes, for the purpose of excluding outliners, and/or the like. In some embodiments, values relating to a same physiological and/or non-physiological parameter obtained via a plurality of wearable devices that are operable engaged with the same subject may be processed to provide a more reliable output. For example, an (e.g., weighted) average of a plurality of values obtained from the corresponding plurality of wearable devices may be provided as an output. In some further examples, the median value may be output by infection detection system 1000. Optionally, rolling averages may be produced as output for a parameter of a certain wearable device. The rolling average obtained from the plurality of wearable devices further processed, e.g., through averaging, to obtain an additional output.

Additional or alternative processing of data values may be employed by any of the plurality of wearable devices, for example, to provide an output that reflects a combined or weighted value of a number of values obtained from the plurality of wearable devices with respect to a certain physiological and/or non-physiological parameter.

According to some embodiments, PNOC analysis engine 1240 may be configured to receive at least one cohort-inclusion criterion defining one or more conditions for associating or inclusion of at least two of a plurality of subjects in a cohort.

The at least one cohort-inclusion criterion may pertain to the location of subjects, travel itinerary, travel conditions, commuting route, age, gender, sex, race, medical background, and/or the like. For example, subjects with identical travel itinerary between a geographical origin and destination may be associated with each other in a cohort. In further example, cluster analysis may be performed by PNOC analysis engine 1240 for associating subjects with a cohort. In a yet further example, age, gender, race and/or other parameters may be considered for inclusion or non-inclusion of one or more subjects into a certain cohort, or for exclusion of one or more subjects presently associated with a cohort.

In some embodiments, infection detection system 1000 may be configured to implement artificial-intelligence functionalities, for example, for associating with or disassociating a subject from a cohort. For example, infection detection system 1000 may receive cohort data, which may be used as training input data for training a machine learning model. The system thus facilitates the generation of cohort training sets for promoting artificial intelligence systems in a variety of subject monitoring and infection detection applications.

In some embodiments, parameter values of subjects already associated with a cohort may be continuously or substantially monitored for determining whether a subject meets the requirements (the at least one “cohort-inclusion criterion”) for remaining included in the cohort. For example, PNOC analysis engine 1240 may monitor physiological and/or non-physiological data related to subjects for retaining those in the cohort which meet the at least one cohort-inclusion criterion; to identify outliers with respect to movement patterns within a geographic area or along a travel journey; and to exclude subjects identified as outliers from the cohort. Furthermore, PNOC analysis engine 1240 may identify subjects that meet the at least one cohort-inclusion criterion and add them to an existing cohort, thereby possibly increasing the number of subjects in a cohort.

Taking into consideration, for example, a subject's journey, commuting route, etc., and further by processing physiological and non-physiological data of a subject along such journey our route, infections may be more reliably detected. For example, by taking into consideration also non-physiological data, the probability of determining false positives may be reduced in comparison to methods and/or system where non-physiological data such as environmental data are not taken into consideration.

In some embodiments, an infection detection criterion is met when a probability of a false-positive determination that a subject is affected by an infectious disease is below a low limit threshold probability. The probability of a false-positive outcome may be reduced, for example, by determining a correlation between physiological parameter values with non-physiological parameter values (e.g., elevated body temperature with physical activity and/or temperature and/or relative humidity of the environment in which the subject is located).

For example, a subject's body temperature may be above average because the subject is carrying heavy luggage and running to timely board a passenger airplane, train and/or the like. In such a scenario, the subject's activity is expected to positively correlate with elevated body temperature, which is thus unlikely to provide a good indication that the subject is suffering from an infectious disease. According to some embodiments, drawbacks regarding comparatively increased probability of false positives when performing single-point-in-time temperature measurement of an individual prior to boarding, for example, an airplane, may thus be reduced or eliminated.

For example, before the time a subject is expected to board a means for mass transportation, data pertaining to the subject may be processed and analyzed to determine whether the subject is suffering from an infectious disease, e.g., to detect such disease before boarding. PNOC analysis engine 1240 may for instance divide a time period that spans from, for example, 1-2 days before a subject boards a means for mass transportation until disembarkation, into various time intervals and consider in the different intervals different physiological and/or non-physiological data values in association with each other, e.g., to determine a degree of relationship (e.g., correlation) between the data values, for determining, based on the degree of relationship, the probability that the subject is suffering from an infectious disease.

For example, a first interval may include a few hours or days before the subject leaves for the journey, second interval may pertain to the journey from the office or home to the airport, and a third interval may be the time the subject is seated in the airplane while at rest. In some examples, intervals may be subdivided into subintervals. For example, in long-haul flights the interval may be divided into sleep time vs time the subject is awake, the time during which a meal is served, etc. For example, the temporal evolution of a plurality of physiological and non-physiological parameter values received by infection detection system 1000 (e.g., through wearable device 1100) can recorded and, due to the time stamps of the stored data, process the data for relating physiological data with non-physiological data such as, for example, humidity and temperature in the airplane, direction of flight to derive an measure of jetlag, the time the meal is served, the time of lights are dimmed in the airplane to facilitate sleeping, and/or the like. As an example, the temperature and humidity levels in an airplane are measured continuously, the times when meals or snacks are served are provided to the system, and the periods during which lights are dimmed for easier sleeping are provided to the system as input.

Considering now, for example, a scenario where a person that was feeling unwell before starting a journey took medication against some health conditions (e.g., to mitigate clinical symptoms indicative of an infection), such as fever-reducing drugs, cough-reducing drugs and/or chill-reducing medication. In such scenario, false-negative identification can occur during the time the medication is effective.

In some embodiments, infection detection system 1000 may also be configured to reduce the number of false-negative outputs, for example, by monitoring the subject for longer time periods than the drugs are effective, such that the re-emergence of the clinical signs indicative of an infection can be recognized. For more accuracy in the diagnosis, analysis of body fluids may be required, with which biochemical signs of infections can be recognized despite the suppression of some clinical signs such as fever,

In some embodiments, the subject's behavior for a certain time period (e.g., a few hours) prior to embarking to a journey may be monitored to identify deviations in physiological characteristics that may provide an indication that the subject is suffering from an infection.

In some embodiments, PNOC analysis engine 1240 may process travel-related data of a person to determine whether a person is subjected to alterations in his/her circadian cycles.

Much of the human physiology exhibits a circadian rhythm, i.e., it shows a periodicity of 24 hours. As an example, considering the expression of the human genes, most of which are also showing a circadian rhythm.

In fact, up to 82 percent of mammalian genes are expressed in a cyclical pattern of highs and lows according to the time of day.

Consequently, a Fourier transform of the temporal evolution of most vital signs will show a strong signal at the frequency of every 24 hours. Accordingly, the following method may be employed for analyzing a circadian rhythm of an individual to identify anomalies in relation to the individual's circadian rhythm:

In an embodiment, the method may include performing Fourier transform of the temporal evolution of a vital sign signal measured over a prolonged time period spanning a number of 24-hours periods (e.g., a week or longer, a month or longer, three months or longer) to obtain a frequency domain representation thereof.

In an embodiment, the vital sign frequency domain representation is analyzed for frequency of every 24 hours of the prolonged time period and may then be compared with the average frequency domain representation of the signal over a plurality of frequencies (“frequency zero”) to derive how strong the signal's 24-hours periodicity is. The above steps may be performed for a plurality of different vital sign signals in the frequency domain.

The method may further include determining whether there is a phase shift between the various vital sign signals with respect to the 24 hours-based frequency, to determine a level of synchronization between the circadian rhythms of the various vital sign signals.

The frequency domain representation of one or more vital sign signals may also be analyzed with respect to the occurrence of higher harmonics, giving additional information about the signal characteristics of the subject's circadian rhythm.

Hence, based on determining a measure of strength of the 24-hours periodicity of a vital sign, and, optionally, the level of synchronization between the circadian rhythms of a plurality of vital sign signals and/or, optionally, higher-harmonic characteristics, circadian signal characteristics (e.g., alterations) may be determined for a subject.

In some examples, the extent of the circadian alterations may be taken into consideration to determine whether or to what extent the person may become jet-lagged. The effect of jetlag on a subject's physiological parameter values may be taken into consideration by PNOC analysis engine 1240 in determining whether the subject meets the at least one infection-detection criterion or not. For example, physiological threshold values indicating an infection may be automatically adapted by PNOC analysis engine 1240 in accordance with an objective measure for measuring the severity to which the person is suffering from jetlag. For example, upper limit thresholds used for defining a physiological parameter value as anomalous may be raised as a result of jetlag. For example, lower limit thresholds used for defining a physiological parameter value as anomalous may be lowered as a result of jetlag. This way, for a jetlagged subject, the bar for identifying physiological parameter values of the subject as anomalous, is raised.

An objective measure for determining whether a subject is jetlagged (e.g., lack of wakefulness) or a severity of jetlag may be based on the subject's body temperature, blood pressure, heart rate, breathing rate, eye movement, EEG signals, hormone levels, physical motor activity, motor responsiveness, etc. For example, slowed reflexes and/responsiveness by a subject compared to the subject's baseline may provide an indication of severity of jetlag.

In some examples, towards the end of the journey of a traveler, all the acquired data is read out of the wearable device's memory. In case the device has a wireless interface, the data can be sent to a processing station wirelessly, for example while the traveler is still wearing the device. The result of the processing can therefore be used once the traveler is leaving the means of transportation. Based on the processing result, e.g., at the exit, the traveler can be told whether there are no symptoms of an infection so that he/she can proceed to the exit, or whether the traveler needs medical attention.

In case the wearable device does not have a wireless interface, the traveler needs to take it off under controlled conditions in a special place, where the device is plugged into a system for downloading and processing the acquired data. The traveler is then told whether there are no symptoms of an infection and the traveler can proceed to the exit, or whether the traveler needs medical attention. The wearable device may be removed under controlled conditions in a special place at a medical checkpoint. There the device may be plugged into a system for downloading and processing the acquired data.

It is noted that the use of the device and method according to embodiments disclosed herein is not limited to travel with public transportation means. Individual travelers, using for example a car, a motorbike, a bicycle or walking on foot can also be monitored for clinical signs of an infection, provided the journey takes an extended time of several ten minutes up to a few days. This requires two medical checkpoints, one at the beginning of a journey or shortly afterwards, and the other one at the end of the journey or shortly before the end.

In some embodiments, at the first medical checkpoint, the traveler is handed out the wearable, and the traveler is instructed how it must be worn and employed. Once it has been assured that the wearable is acquiring all intended vital signs, for example by reading out all its sensors wirelessly during a few seconds, the traveler can start or continue her journey, during which the wearable must be worn at all times at the body location(s) and in the manner prescribed by the authorized medical personnel. At the second medical checkpoint, all the acquired data is read out of the wearable device's memory. In case the device has a wireless interface, the data can be sent to a processing station wirelessly, for example while the traveler is still wearing the device. The result of the processing can therefore be used for further analysis before the traveler ends her journey. Thus, the subject can be told at the second medical checkpoint whether there are no symptoms of an infection and the traveler can proceed, or whether the traveler needs medical attention.

In some embodiments, the processing of the vital signs occurs in the following way: Periods of rest are identified, based on the measurements of the corresponding activity sensors of the wearable device. For these time periods, environmental conditions are obtained from external sources, such as temperature or humidity, and special events are identified, i.e., when meals are served, or lights are dimmed for better sleeping. This information is employed to determine individual baseline and/or reference values of the observed vital signs that are indicative of an infection. As mentioned above, this includes increased temperature (fever), increased sweating, coughing, shortness of breath, sneezing, shivering, increased heart rate, hypotension (lowered blood pressure).

In some embodiments, the individual baseline values of the various clinical signs of infection are analyzed as a function of time during the period of the travel. The goal is to identify a trend during the travel period: When a baseline values of the various vital signs indicative of an infection remain constant or are decreasing, the probability is high that the traveler is not infected. However, when these clinical signs of an infection are increasing, then the traveler needs medical attention, for example by taking her blood and checking for the presence of bacteria or viruses causing the suspected infection.

According to some embodiments, the device and method according can also be used for monitoring people who were put into quarantine because of a suspected and not yet microbiologically confirmed infection. A subject's vital signs that are indicative of an infection can be continuously monitored, and temporal trends can be easily recognized without the regular intervention of trained or medical personnel. Consequently, persons showing signs of an emerging infection can be identified earlier than with state-of-the-art methods, and these threatened persons can be taken into medical care.

Once people have obtained medical treatment for their particular infection, for example at a hospital, at a doctor's practice or at a special point of care, it is desirable to check the success of the given treatment after the discharge of these persons. This can be accomplished by supplying each of these treated subjects, also called out-patients, with a wearable device according to embodiments, for the continuous acquisition of their vital signs that are indicative of infection, and by analyzing this data periodically according to the method described above. Temporal trends can be easily recognized without the regular intervention of medical personnel. As a consequence, out-patients showing signs of unsatisfactory health progress or failing treatment can be identified rapidly, and these out-patients can be called back into medical care.

The device and method, according to some embodiments, can also be used to estimate the spread of an endemic or even pandemic disease: A small fraction of the population, for example 1%, is supplied with the wearable device that must be worn during a few days. During this period, a multitude of vital signs of the user are monitored as described above, in order to detect signs of an existing or emerging infection. The spread of an epidemic or pandemic disease can then be estimated as the fraction of users showing clinical signs of an infection divided by the total number of subjects that have been wearing the device.

In some embodiments, based on the data gathered about at least one subject in a cohort, at least one other subject of the same cohort may be notified about the probability to contract an infectious disease from other subjects in the cohort. For example, at least one first subject of a plurality of subjects traveling in a train carriage may exhibit clinical signs relating to an infectious disease, whereas at least one second subject traveling in the same train carriage may not exhibit clinical signs relating to an infectious disease. However, the at least one second subject may be warned that he is traveling with passengers which are likely considered to have contracted an infectious disease, allowing separating the at least one first and second passengers from one another for additional medical examination.

In some embodiments, the location may pertain to the subject's location within a world reference frame. In some embodiments, the location of a subject may pertain to different areas within a building and/or transportation means used by the subject. For example, different train carriages and/or different areas (e.g., floors) and/or departments of a cruise ship and/or building may be associated with different cohorts, non-physiological conditions (air-conditioning parameter values), and/or the like. For instance, a hospital and/or office building may be divided into different areas, based on the department assigned to each of the areas. The system may be configured to monitor movement of one or more subjects in a building and/or means of transportation to determine which area is occupied by which subject and/or cohort member and/or cohort, and further, to detect and/or prevent cross-contamination between objects of different departments and/or areas of a building and/or transportation means to reduce or eliminate the risk that one or more subjects in the building and/or transportation means contract an infection.

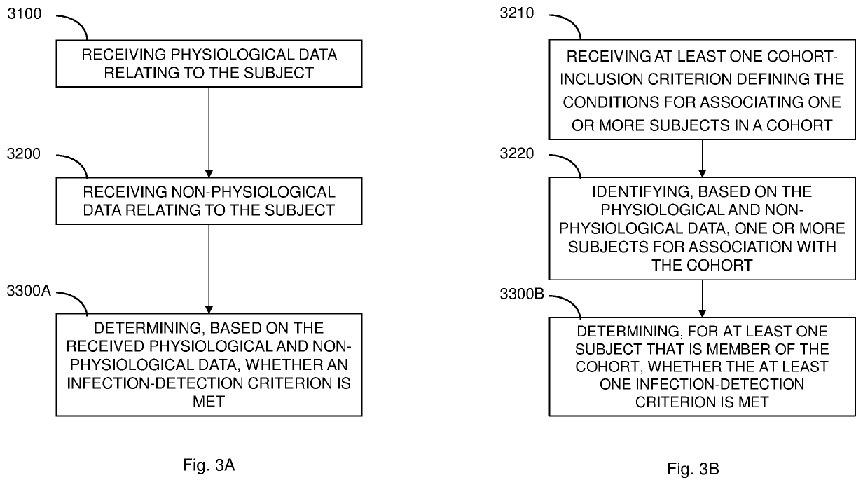

Further reference is made to FIG. 3A. According to some embodiments, a method for determining (e.g., a probability) that a subject is physiologically affected by (e.g., has contracted) an infectious disease, includes receiving physiological data relating to the subject (block 3100). the method may further include receiving non-physiological data relating to the subject (block 3200). the method may additionally include determining, based on the received physiological and non-physiological data, whether an infection-detection criterion is met (block 3300A).

Further referring to FIG. 3B, the method may include, prior to determining whether an infection-detection criterion is met, receiving at least one cohort-inclusion criterion defining the conditions for associating one or more subjects in a cohort (block 3210). the method may further include identifying, based on the physiological and non-physiological data, one or more subjects for association with the cohort (block 3220) and determining, for at least one subject that is member of the cohort, whether the at least one infection-detection criterion is met (block 3300B).

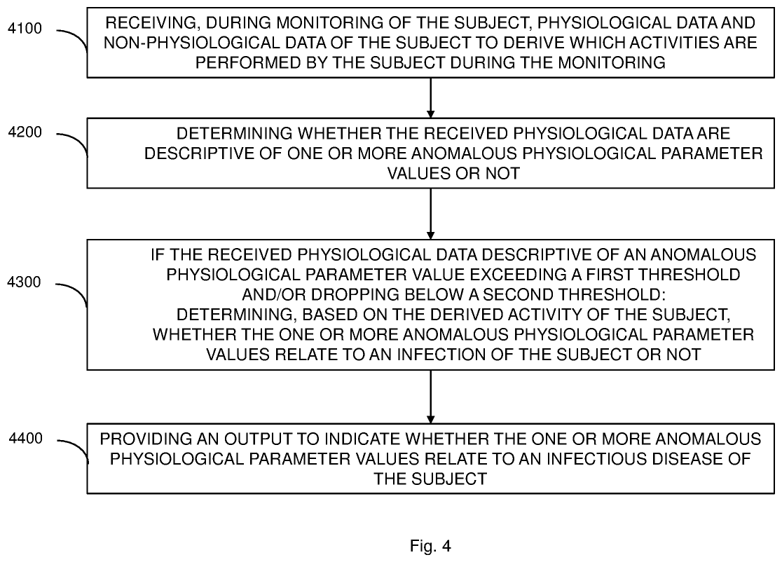

Further referring to FIG. 4, a method may in some embodiments include receiving, during monitoring of the subject, physiological data and non-physiological data of the subject to derive which activities are performed by the subject during the monitoring (Block 4100).

In some embodiments, the method may further include determining whether the received physiological data are descriptive of one or more anomalous physiological parameter values or not (block 4200).

In some embodiments, the method may also include, if the received physiological data descriptive of an anomalous physiological parameter value exceeding a first threshold and/or dropping below a second threshold: determining, based on the derived activity of the subject, whether the one or more anomalous physiological parameter values relate to an infection of the subject or not (block 4300).

In some embodiments, the method may include providing an output to indicate whether the one or more anomalous physiological parameter values relate to an infectious disease of the subject or not (block 4400).

### ADDITIONAL EXAMPLES

Example 1 pertains to an infection detection system that is configured, for example, to detect and/or identify an infection or infectious disease in a subject and/or a type of infection or infectious disease, and/or configured to determine the probability that the subject is affected by an infection or by a certain type of infection. The system may comprise:
a memory and a processor, wherein the memory and the processor are configured to enable the system to perform the following:
receiving physiological data descriptive of physiological parameter values of a subject;
receiving non-physiological data relating to the subject; and
determining, based on the received physiological data and the non-physiological data, whether at least one infection-detection criterion is met.
Example 2 includes the subject matter of example 1 and, optionally, wherein the non-physiological data relates to at least one activity pursued by the subject.
Example 3 includes the subject matter of Example 1 and/or Example 2 and, optionally, wherein the system is configured to provide an output that indicates whether the subject is affected by an infectious disease.
Example 4 includes the subject matter of any one or more of the Examples 1 to 3 and, optionally, wherein non-physiological data pertains to a location of the subject (e.g., within a geographic area, a type of vehicle used by the subject for travelling, and/or the like).
Example 5 includes the subject matter of any one or more of the Examples 1 to 4 and, optionally, wherein the non-physiological data pertains to and/or is descriptive of a travel itinerary of the subject.
Example 6 includes the subject matter of any one or more of the Examples 1 to 5 and, optionally, wherein the non-physiological data pertains to environmental conditions in which the subject is located.
Example 7 includes the subject matter of any one or more of the Examples 1 to 6 and, optionally, a wearable device comprising at least one sensor, wherein physiological data and/or non-physiological data are received at the system from the at least one sensor.
Example 8 includes the subject matter of any one or more of the Examples 1 to 7 and, optionally, wherein the system receives physiological data and/or non-physiological data from databases. In some examples, the databases may be external to the system. In some embodiments, data may be pre-stored in the system.
Example 9 includes the subject matter of any one or more of the Examples 1 to 8 and, optionally, wherein the system is further configured to:
receive at least one cohort-inclusion criterion defining the conditions for associating one or more subjects in a cohort;
identify, based on the physiological and non-physiological data, one or more subjects meeting the at least one cohort-inclusion criterion for association of at least one subject of the one or more subjects with the cohort; and
determine, for at least one subject that is member of the cohort, whether the at least one infection-detection criterion is met.
Example 10 pertains to a method for detecting and/or identifying an infection or infectious disease in a subject and/or a type of infection or infectious disease, and/or to determining a probability that the subject is affected by an infection or by a certain type of infection. The method may comprise:
receiving physiological data descriptive of physiological parameter values of a subject;
receiving non-physiological data relating to the subject; and
determining, based on the received physiological data and the non-physiological data, whether at least one infection-detection criterion is met.
Example 11 includes the subject matter of example 10 and, optionally, wherein the non-physiological data relates to at least one activity pursued by the subject.
Example 12 includes the subject matter of any one or more of the Examples 10 to 11 and, optionally, further comprising providing an output that indicates whether the subject is affected by an infectious disease.
Example 13 includes the subject matter of any one or more of the Examples 10 to 12 and, optionally, wherein non-physiological data pertains to the location of the subject or to a geographic area in which the subject is presently or expected to be located within a future time interval.
Example 14 includes the subject matter of any one or more of the Examples 10 to 13 and, optionally, wherein the non-physiological data pertains to and/or is descriptive of a travel itinerary of the subject
Example 15 includes the subject matter of any one or more of the Examples 10 to 14 and, optionally, wherein the non-physiological data pertains to environmental conditions in which the subject is located.
Example 16 includes the subject matter of any one or more of the Examples 10 to 15 and, optionally, wherein physiological data and/or non-physiological data are received at an infection detection system from the at least one sensor of one or more wearable devices worn by the subject.
Example 17 includes the subject matter of any one or more of the Examples 10 to 16 and, optionally, wherein physiological data and/or non-physiological data are received from databases. In some examples, the databases are external to the infection detection system.
Example 18 includes the subject matter of any one or more of the Examples 10 to 17 and, optionally:
receiving at least one cohort-inclusion criterion defining the conditions for associating one or more subjects in a cohort;
identifying, based on the physiological and non-physiological data, one or more subjects for association with the cohort; and
determining, for at least one subject that is member of the cohort, whether the at least one infection-detection criterion is met.
Example 19 includes a computer program product comprising program instructions for the execution of a method comprising:
receiving physiological data descriptive of physiological parameter values of a subject; receiving non-physiological data relating to the subject; and
determining, based on the received physiological data and the non-physiological data, whether at least one infection-detection criterion is met.
Example 20 includes the subject matter of example 19 and, optionally, wherein the non-physiological data relates to at least one activity pursued by the subject.
Example 21 includes the subject matter of examples 19 and/or 20 and, optionally, providing an output that indicates whether the subject is affected by an infectious disease.
Example 22 includes the subject matter of any one or more of the Examples 19 to 21 and, optionally, wherein non-physiological data pertains to the location of the subject within a geographic area.
Example 23 includes the subject matter of any one or more of the Examples 19 to 22 and, optionally, wherein the non-physiological data pertains to and/or is descriptive of the subject's travel itinerary.
Example 24 includes the subject matter of any one or more of the Examples 19 to 23 and, optionally, wherein the non-physiological data pertains to environmental conditions in which the subject is located.
Example 25 includes the subject matter of any one or more of the Examples 19 to 24 and, optionally, wherein physiological data and/or non-physiological data are received at an infection detection system from the at least one sensor of a wearable device.
Example 26 includes the subject matter of any one or more of the Examples 19 to 25 and, optionally, wherein physiological data and/or non-physiological data are received from databases which are external to the infection detection system.
Example 27 includes the subject matter of any one or more of the Examples 19 to 26 and, optionally,
receiving at least one cohort-inclusion criterion defining the conditions for associating one or more subjects with each other in a cohort;
identifying, based on the physiological and non-physiological data, one or more subjects for association with the cohort; and
determining, for at least one subject that is member of the cohort, whether the at least one infection-detection criterion is met.
Example 28 pertains to a device for the early detection of infections, in particular fast-spreading infections that could develop into pandemics, consisting of a wearable device containing several sensor types capable of the continuous measurement of a multitude of a user's vital signs, wherein the vital signs are related to the clinical signs of an infection, so that when the wearable is used for extended times from tens of minutes to several days, preferentially during the time of long-distance travel, it is possible to exclude several non-specific influences on the measurements such as, for example, external conditions during the measurement (e.g. temperature and humidity), individual activity level of a user, individual baselines of the measured vital signs, the individual circadian rhythms of a user, and the individual periodic variations of body temperature of a female user during her menstrual cycle.
Example 29 pertains to an infection detection system comprises a memory for storing software code; and a processor configured to execute software code stored in the memory to enable the system to perform the following:
receiving, during monitoring of the subject, physiological data and non-physiological data of the subject to derive which activities are performed by the subject during the monitoring; determining whether the received physiological data are descriptive of one or more anomalous physiological parameter values or not.
and, when the received physiological data descriptive of an anomalous physiological parameter value exceeding a first threshold or is below a second threshold;
determining, based on the derived activity of the subject, whether the one or more anomalous physiological parameter values relate to an infection of the subject or not; and
providing an output to indicate whether the one or more anomalous physiological parameter values relate to an infectious disease of the subject. Optionally, a physiological parameter value may be defined as anomalous if it exceeds a certain threshold for a predetermined time period or if it is below a certain threshold for an (e.g., predetermined) time period.

In some examples, the non-physiological data pertains to and/or is descriptive of a travel itinerary of the subject.

In some examples, the system receives initial physiological data and/or initial non-physiological data from databases which are external to the infection detection system.

In some examples, the system is configured to perform the following:
receiving at least one cohort-inclusion criterion defining the conditions for associating one or more subjects in a cohort;
identifying, based on the physiological and non-physiological data, one or more subjects for association with the cohort; and
determining, for at least one subject, that is member of the cohort,
determining, whether the received physiological data of the at least one subject are descriptive of one or more anomalous physiological parameter values of the at least one subject, or not;
determining, based on the derived activity of the at least one subject in comparison with at least one other subject of the cohort, whether the one or more anomalous physiological parameter values of the at least one subject relate to an infection of the subject or not. In that case, it may be considered that an infection-detection criterion is met.

Optionally, an output may be provided indicating whether the one or more anomalous physiological parameter values relate to an infectious disease of the subject.

In some examples, a method for identifying an infectious disease in a subject comprises: receiving, during monitoring of the subject, physiological data and non-physiological data of the subject to derive which activities are performed by the subject during the monitoring; determining whether the received physiological data are descriptive of one or more anomalous physiological parameter values or not; and, when the received physiological data descriptive of an anomalous physiological parameter value exceeds a first threshold and/or is below a second threshold: determining, for instance based on the derived activity of the subject, whether the one or more anomalous physiological parameter values relate to an infection of the subject or not; and, optionally, providing an output to indicate whether the one or more anomalous physiological parameter values relate to an infectious disease of the subject.

In some examples, the non-physiological data pertains to and/or is descriptive of a travel itinerary of the subject.

In some examples, the non-physiological data pertains to environmental conditions in which the subject is located.

In some examples, a method may include receiving, from a plurality of subjects, physiological and non-physiological data of the plurality of subjects including, for example, a location of the plurality of subjects, and identifying at least two subjects of the plurality of subjects that are located in a same geographical area. The method may further include associating the at least two subjects that are located in the same geographical area with a cohort; and receiving environmental data descriptive of environmental characteristics of the geographic area including temperature measurements. The method may further include determining whether the received updated physiological data relate to an anomalous physiological parameter value or not; and deriving, based on the received physiological and non-physiological data, an activity performed by at least one first subject and at least one second subject of the at least two subjects performed in the geographical area.

The method may additionally include analyzing the activity of the at least one first subject and of at least one second subject. The analyzing may include comparing the activity of the at least one first subject with the activity of at least one second subject. The analyzing may be performed by correlating activity of the at least one first subject and of at least one second subject, and of non-physiological data of the at least one first subject and the at least one second subject.

When the received updated physiological data parameter value is indicative of an anomalous physiological parameter value, the method may include identifying, for the anomalous physiological parameter value, e.g., based on the analysis (e.g., comparison or correlation) of the performed activities, the physiological data, and/or the environmental characteristics in the geographic area, a false-positive diagnosis that the at least one first and/or second subjects are affected by an infectious disease. An anomalous physiological parameter value may be labeled or identified as being conducive of a false-positive diagnosis if the determined correlation is above a high coefficient threshold. On the other hand, an anomalous physiological parameter value may be identified as being indicative of an infectious disease of the subject if the determined correlation is below a low coefficient threshold.

In some examples, when the anomalous physiological parameter value is not a false-positive diagnosis that the at least one first and/or second subject is affected by an infectious disease: the method may further comprise providing an output indicating that the corresponding subject is affected by an infectious disease. In that case, it may be considered that an infection-detection criterion is met.

In some examples, a computer program product comprising program instructions for the execution of a method comprising: receiving initial physiological data descriptive of physiological parameter values of a subject; receiving initial non-physiological data relating to the subject; and receiving, during monitoring of the subject, updated physiological data and updated non-physiological data of the subject; deriving, based on the updated physiological data and non-physiological data, which activities are performed by the subject during the monitoring;

determining whether the received updated physiological data relate to an anomalous physiological parameter value or not; and, when the received updated physiological data parameter value is indicative of an anomalous physiological parameter value: determining, based on the derived activity of the subject, whether the updated physiological data relate to an infection of the subject or not; and, optionally, providing an output to indicate whether the anomalous physiological parameter value relates to an infectious disease of the subject.

In some examples, physiological data and/or non-physiological data are received at an infection detection system from at least one sensor of a wearable device.

In some examples, the computer program product may further be configured to perform: receiving, from a plurality of subjects, physiological and non-physiological data of the plurality of subjects including a location of the plurality of subjects,
identifying at least two subjects of the plurality of subjects which are located in a same geographical area;
associating the at least two subjects that are located in the same geographical area with a cohort;
receiving environmental characteristic values of the geographic area, deriving, based on the received physiological and non-physiological data, an activity performed by at least one first subject and at least one second subject of the at least two subjects performed in the geographical area;
comparing the activity of the at least one first subject with the activity of at least one second subject;
identifying, when received physiological data is descriptive of an anormal physiological parameter value, based on the comparison of the performed activities and the environmental characteristics in the geographic area, a false-positive diagnostics that the at least one first and/or second subjects are affected by an infectious disease; and
providing an output indicating that the anomalous physiological parameter value is a false-positive value regarding an infectious disease.

In some examples, the identification of the false-positive diagnostics comprises:
calculating, for the at least one subject, a correlation coefficient between the derived activities, environmental characteristics and the anomalous physiological parameter value; and
when the correlation exceeds a high-correlation threshold, providing an output indicating that the at least one first and/or second subject for which an anomalous physiological parameter value is received, is not affected by an infectious disease.

In some examples, a method for identifying an infectious disease in a subject comprises:
receiving, from a plurality of subjects, physiological and non-physiological data of the plurality of subjects including a location of the plurality of subjects,
identifying at least two subjects of the plurality of subjects which are located in a same geographical area;
associating the at least two subjects that are located in the same geographical area with a cohort;
monitoring environmental characteristics of the geographic area,
deriving, based on the received physiological and non-physiological data, an activity performed by at least one first subject and at least one second subject of the at least two subjects performed in the geographical area;
comparing the activity of the at least one first subject with the activity of at least one second subject; and
determining, for the at least one first subject and/or the at least one second subject, whether a received physiological data parameter value is an anomalous physiological parameter value or not;
automatically determining, based on the comparison of the performed activities and the environmental characteristics in the geographic area, with respect to the anomalous physiological parameter value, a probability or likelihood ratio of a false-positive diagnostics that the at least one first and/or second subjects are affected by an infectious disease.

In some examples, the method may include and/or the system may be configured to perform:
distinguishing, for example, based on the comparison of the performed activities and the environmental characteristics in the geographic area, between anomalous physiological parameter values that relate (e.g., correlate) with an event or state that the at least one first and/or second subjects are affected by an infectious disease, and anomalous physiological parameter values that do not relate (e.g., correlate) with an event or state that the at least one first and/or second subjects are affected by an infectious disease.

In some examples, deriving an activity of the least one first subject and the at least one second subject includes determining whether the at least one first and/or second subject are in a resting state or whether they perform a strenuous activity. This may for example be accomplished by monitoring pulse, heart rate, sweat rate, body movements (e.g., determining whether the person is resting, or performing a sports activity, physical labor), and/or the like. When the system determines that physiological data descriptive of anomalous physical values such as increased pulse, heart rate, sweat rate, and body temperature (e.g., in an environment of elevated temperature) correlates with exercise and/or physical labor under harsh conditions, then the system may determine that the subject is performing strenuous activity, and label such anomalous physical values as not related to an infectious disease of the subject. On the other hand, when the system determines that physiological data descriptive of anomalous physical values such as increased pulse, heart rate, sweat rate, and body temperature (e.g., in an environment of elevated temperature) does not correlate with the subject's physical activity then the system may determine that the subject is in a resting state and label physical values identified as anomalous being related to an infectious disease of the subject.

In some examples, an infection detection system comprises: a memory; and a processor, wherein the memory and the processor are configured to enable the system to perform the following:
receiving, from a plurality of subjects, physiological and non-physiological data of the plurality of subjects including a location of the plurality of subjects acquired, e.g., by a wearable sensor system,
identifying at least two subjects of the plurality of subjects which are located in a same geographical area;
associating the at least two subjects that are located in the same geographical area with a cohort;
monitoring environmental characteristics of the geographic area,
deriving, based on the received physiological and non-physiological data, an activity performed by at least one first subject and at least one second subject of the at least two subjects performed in the geographical area;
comparing the activity of the at least one first subject with the activity of at least one second subject; and
automatically determining, based on the comparison of the performed activities and the environmental characteristics in the geographic area, a probability of a false-positive diagnostics that the at least one first and/or second subjects are affected by an infectious disease.

In some examples, the deriving of the least one first subject and the at least one second subject includes determining whether the at least one first and/or second subject is in a resting state or whether the at least one first and/or second subject performs a strenuous activity.

Embodiments may also pertain to a device for the early detection of infections, in particular fast-spreading infections that could develop into pandemics, consisting of a wearable device containing several sensor types capable of the continuous measurement of a multitude of a user's vital signs, wherein the vital signs are related to the clinical signs of an infection, so that when the wearable is used for extended times from tens of minutes to several days, preferentially during the time of long-distance travel, it is possible to exclude several non-specific influences on the measurements, such as external conditions during the measurement (e.g. temperature and humidity), individual activity level of a user, individual baselines of the measured vital signs, the individual circadian rhythms of a user, and the individual periodic variations of body temperature of a female user during her menstrual cycle.

It is important to note that the methods described herein and illustrated in the accompanying diagrams shall not be construed in a limiting manner. For example, methods described herein may include additional or even fewer processes or operations in comparison to what is described herein and/or illustrated in the diagrams. In addition, method steps are not necessarily limited to the chronological order as illustrated and described herein.

Any digital computer system, unit, device, module and/or engine exemplified herein can be configured or otherwise programmed to implement a method disclosed herein, and to the extent that the system, module and/or engine is configured to implement such a method, it is within the scope and spirit of the disclosure. Once the system, module and/or engine are programmed to perform particular functions pursuant to computer readable and executable instructions from program software that implements a method disclosed herein, it in effect becomes a special purpose computer particular to embodiments of the method disclosed herein. The methods and/or processes disclosed herein may be implemented as a computer program product that may be tangibly embodied in an information carrier including, for example, in a non-transitory tangible computer-readable and/or non-transitory tangible machine-readable storage device. The computer program product may directly loadable into an internal memory of a digital computer, comprising software code portions for performing the methods and/or processes as disclosed herein.

The methods and/or processes disclosed herein may be implemented as a computer program that may be intangibly embodied by a computer readable signal medium. A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a non-transitory computer or machine-readable storage device and that can communicate, propagate, or transport a program for use by or in connection with apparatuses, systems, platforms, methods, operations and/or processes discussed herein.

The terms “non-transitory computer-readable storage device” and “non-transitory machine-readable storage device” encompass distribution media, intermediate storage media, execution memory of a computer, and any other medium or device capable of storing for later reading by a computer program implementing embodiments of a method disclosed herein. A computer program product can be deployed to be executed on one computer or on multiple computers at one site or distributed across multiple sites and interconnected by one or more communication networks.

These computer readable and executable instructions may be provided to a processor of a general-purpose computer, a special-purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable and executable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable and executable instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The term “engine” may comprise one or more computer modules, wherein a module may be a self-contained hardware and/or software component that interfaces with a larger system. A module may comprise a machine or machines executable instructions. A module may be embodied by a circuit or a controller programmed to cause the system to implement the method, process and/or operation as disclosed herein. For example, a module may be implemented as a hardware circuit comprising, e.g., custom Very-Large-Scale-Integrated (VLSI) circuits or gate arrays, an application-specific integrated circuit (ASIC), off-the-shelf semiconductors such as logic chips, transistors, and/or other discrete components. A module may also be implemented in programmable hardware devices such as field programmable gate arrays, programmable array logic, programmable logic devices and/or the like.

The term “random” also encompasses the meaning of the term “substantially randomly” or “pseudo-randomly”.

The expression “real-time” as used herein generally refers to the updating of information based on received data, at essentially the same rate as the data is received, for instance, without user-noticeable judder, latency, or lag.

In the discussion, unless otherwise stated, adjectives such as “substantially” and “about” that modify a condition or relationship characteristic of a feature or features of an embodiment, are to be understood to mean that the condition or characteristic is defined to within tolerances that are acceptable for operation of the embodiment for an application for which it is intended.

Unless otherwise specified, the terms “substantially”, “′about” and/or “close” with respect to a magnitude or a numerical value may imply to be within an inclusive range of −10% to +10% of the respective magnitude or value.

“Coupled with” can mean indirectly or directly “coupled with”.

It is important to note that the method may include is not limited to those diagrams or to the corresponding descriptions. For example, the method may include additional or even fewer processes or operations in comparison to what is described in the figures. In addition, embodiments of the method are not necessarily limited to the chronological order as illustrated and described herein.

Discussions herein utilizing terms such as, for example, “processing”, “computing”, “calculating”, “determining”, “establishing”, “analyzing”, “checking”, “estimating”, “deriving”, “selecting”, “inferring” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes. The term “determining” may, where applicable, also refer to “heuristically determining”.

It should be noted that where an embodiment refers to a condition of “above a threshold”, this should not be construed as excluding an embodiment referring to a condition of “equal or above a threshold”. Analogously, where an embodiment refers to a condition “below a threshold”, this should not be construed as excluding an embodiment referring to a condition “equal or below a threshold”. It is clear that should a condition be interpreted as being fulfilled if the value of a given parameter is above a threshold, then the same condition is considered as not being fulfilled if the value of the given parameter is equal or below the given threshold. Conversely, should a condition be interpreted as being fulfilled if the value of a given parameter is equal or above a threshold, then the same condition is considered as not being fulfilled if the value of the given parameter is below (and only below) the given threshold.

It should be understood that where the claims or specification refer to “a” or “an” element and/or feature, such reference is not to be construed as there being only one of that element. Hence, reference to “an element” or “at least one element” for instance may also encompass “one or more elements”.

Terms used in the singular shall also include the plural, except where expressly otherwise stated or where the context otherwise requires.

In the description and claims of the present application, each of the verbs, “comprise” “include” and “have”, and conjugates thereof, are used to indicate that the data portion or data portions of the verb are not necessarily a complete listing of components, elements or parts of the subject or subjects of the verb.

Unless otherwise stated, the use of the expression “and/or” between the last two members of a list of options for selection indicates that a selection of one or more of the listed options is appropriate and may be made. Further, the use of the expression “and/or” may be used interchangeably with the expressions “at least one of the following”, “any one of the following” or “one or more of the following”, followed by a listing of the various options.

As used herein, the phrase “A, B, C, or any combination of the aforesaid” should be interpreted as meaning all of the following: (i) A or B or C or any combination of A, B, and C, (ii) at least one of A, B, and C; (iii) A, and/or B and/or C, and (iv) A, B and/or C. Where appropriate, the phrase A, B and/or C can be interpreted as meaning A, B or C. The phrase A, B or C should be interpreted as meaning “selected from the group consisting of A, B and C”. This concept is illustrated for three elements (i.e., A, B, C), but extends to fewer and greater numbers of elements (e.g., A, B, C, D, etc.).

It is appreciated that certain features of the invention, which are, for clarity, described in the context of separate embodiments or example, may also be provided in combination in a single embodiment. Conversely, various features of the invention, which are, for brevity, described in the context of a single embodiment, example and/or option, may also be provided separately or in any suitable sub-combination or as suitable in any other described embodiment, example or option of the invention. Certain features described in the context of various embodiments, examples and/or optional implementation are not to be considered essential features of those embodiments, unless the embodiment, example and/or optional implementation is inoperative without those elements.

It is noted that the terms “in some embodiments”, “according to some embodiments”, “for example”, “e.g.”, “for instance” and “optionally” may herein be used interchangeably.

The number of elements shown in the Figures should by no means be construed as limiting and is for illustrative purposes only.

It is noted that the terms “operable to” can encompass the meaning of the term “modified or configured to”. In other words, a machine “operable to” perform a task can in some embodiments, embrace a mere capability (e.g., “modified”) to perform the function and, in some other embodiments, a machine that is actually made (e.g., “configured”) to perform the function.

Throughout this application, various embodiments may be presented in and/or relate to a range format. It should be understood that the description in range format is merely for convenience and brevity and should not be construed as an inflexible limitation on the scope of the embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

While the invention has been described with respect to a limited number of embodiments, these should not be construed as limitations on the scope of the invention, but rather as exemplifications of some of the embodiments.

What is claimed is:

1. An infection detection system comprising:
a memory for storing software code; and
a processor configured to executed software code stored in the memory to enable the system to perform the following:
receiving, during monitoring of the subject, physiological data and non-physiological data of the subject to derive which activities are performed by the subject during the monitoring;
determining whether the received physiological data are descriptive of one or more anomalous physiological parameter values or not;
and, when or if the received physiological data descriptive of an anomalous physiological parameter value exceeding a first threshold and/or dropping below a second threshold;
determining, based on the derived activity of the subject, whether the one or more anomalous physiological parameter values relate to an infection of the subject or not; and
providing an output to indicate whether the one or more anomalous physiological parameter values relate to an infectious disease of the subject.

2. The infection detection system of claim 1, wherein the non-physiological data pertains to and/or is descriptive of a travel itinerary of the subject.

3. The infection detection system of claim wherein the non-physiological data pertains to environmental conditions in which the subject is located.

4. The infection detection system of claim 1, further comprising:
a wearable device comprising at least one sensor,
wherein physiological data and/or non-physiological data are received at the system from the at least one sensor.

5. The infection detection system of claim 1, wherein the system receives initial physiological data and/or initial non-physiological data from databases which are external to the infection detection system.

6. The infection detection system claim 1, further configured to:
receiving at least one cohort-inclusion criterion defining the conditions for associating one or more subjects in a cohort;
identifying, based on the physiological and non-physiological data, one or more subjects for association with the cohort; and
determining, for at least one subject that is member of the cohort, determining, whether the received physiological data of the at least one subject are descriptive of one or more anomalous physiological parameter values of the at least one subject, or not;
determining, based on the derived activity of the at least one subject in comparison with at least one other subject of the cohort whether the one or more anomalous physiological parameter values of the at least one subject relate to an infection of the subject or not; and
providing an output to indicate whether the one or more anomalous physiological parameter values relate to an infectious disease of the subject.

7. A method for identifying an infectious disease in a subject, the method comprising:
receiving, during monitoring of the subject, physiological data and non-physiological data of the subject to derive which activities are performed by the subject during the monitoring;
determining whether the received physiological data are descriptive of one or more anomalous physiological parameter values or not;
and, when the received physiological data descriptive of an anomalous physiological parameter value exceeding a first threshold and/or dropping below a second threshold;
determining, based on the derived activity of the subject, whether the one or more anomalous physiological parameter values relate to an infection of the subject or not; and
providing an output to indicate whether the one or more anomalous physiological parameter values relate to an infectious disease of the subject.

8. The method of claim 7, wherein the non-physiological data pertains to and/or is descriptive of a travel itinerary of the subject.

9. The method of claim 7, wherein the non-physiological data pertains to environmental conditions in which the subject is located.

10. The method of claim 7, wherein physiological data and/or non-physiological data are received at an infection detection system from at least one sensor of a wearable device.

11. The method of claim 10, wherein physiological data and/or non-physiological data are received from databases which are external to the infection detection system.

12. The method of claim 7, further comprising:
receiving, from a plurality of subjects, physiological and non-physiological data of the plurality of subjects including a location of the plurality of subjects,
identifying at least two subjects of the plurality of subjects that are located in a same geographical area;
associating the at least two subjects that are located in the same geographical area with a cohort;
receiving environmental data descriptive of environmental characteristics of the geographic area including temperature measurements,
determining whether the received updated physiological data relate to an anomalous physiological parameter value or not;
deriving, based on the received physiological and non-physiological data, an activity performed by at least one first subject and at least one second subject of the at least two subjects performed in the geographical area;
comparing the activity of the at least one first subject with the activity of at least one second subject; and
and, when the received updated physiological data parameter value is indicative of an anomalous physiological parameter value:
identifying for the anomalous physiological parameter value, based on the comparison of the performed activities and the environmental characteristics in the geographic area, a false-positive diagnostics that the at least one first and/or second subjects are affected by an infectious disease.

13. The method of claim 12, further comprising:
when the anomalous physiological parameter value does not provide a false-positive indication that the at least one first and/or second subject is affected by an infectious disease:
providing an output indicating that the corresponding subject is affected by an infectious disease.

14. The method of claim 7, further comprising:
receiving initial physiological data descriptive of physiological parameter values of a subject;
receiving initial non-physiological data relating to the subject; and
receiving, during monitoring of the subject, updated physiological data and updated non-physiological data of the subject
deriving, based on the updated physiological data and non-physiological data, which activities are performed by the subject during the monitoring;
determining whether the received updated physiological data relate to an anomalous physiological parameter value or not;
and, when the received updated physiological data parameter value is indicative of an anomalous physiological parameter value:
determining, based on the derived activity of the subject, whether the updated physiological data relate to an infection of the subject or not; and
providing an output to indicate whether the anomalous physiological parameter value relates to an infectious disease of the subject.

15. The method of claim 14, wherein the non-physiological data pertains to and/or is descriptive of the subject's travel itinerary.

16. The method of claim 14, wherein the non-physiological data pertains to environmental conditions in which the subject is located.

17. The method of claim 14, wherein physiological data and/or non-physiological data are received at an infection detection system from at least one sensor of a wearable device.

18. (canceled)

19. (canceled)

20. (canceled)

21. (canceled)

22. (canceled)

23. An infection detection system comprising:
a memory; and
a processor, wherein the memory and the processor are configured to enable the system to perform the following:
receiving, from a plurality of subjects, physiological and non-physiological data of the plurality of subjects including a location of the plurality of subjects,
identifying at least two subjects of the plurality of subjects which are located in a same geographical area;
associating the at least two subjects that are located in the same geographical area with a cohort;
monitoring environmental characteristics of the geographic area,
deriving, based on the received physiological and non-physiological data, an activity performed by at least one first subject and at least one second subject of the at least two subjects performed in the geographical area;
comparing the activity of the at least one first subject with the activity of at least one second subject; and
automatically determining, based on the comparison of the performed activities and the environmental characteristics in the geographic area, a likelihood ratio of a false-positive diagnostics that the at least one first and/or second subjects are affected by an infectious disease.

24. The system of claim 23, wherein the deriving of the least one first subject and the at least one second subject includes determining whether the at least one first and/or second subject is in a resting state or whether the at least one first and/or second subject performs a strenuous activity.

25. The system of claim 23, further configured to
wherein the automatically determining of the likelihood ratio of the false-positive diagnostic also takes into consideration one the following parameter values:
the individual circadian rhythms of a user, the individual periodic variations of body temperature of a female user during her menstrual cycle, or both.
